(19) United States
(12) Patent Application Publication
(10) Pub. No.: US 2017/0014386 A1
(43) Pub. Date: Jan. 19, 2017

(54) Treatment of Multidrug-Resistant Nephrotic Syndrome (MDR-NS) in Children

(71) Applicant: AbbVie, Inc., North Chicago, IL (US)

(72) Inventors: John J. Brennan, Libertyville, IL (US); Franz Schaefer, Heidelberg (DE); Melissa E. Wigderson, North Chicago, IL (US)

(21) Appl. No.: 15/211,781

(22) Filed: Jul. 15, 2016

Related U.S. Application Data
(60) Provisional application No. 62/193,410, filed on Jul. 16, 2015.
(60) Provisional application No. 62/257,536, filed on Nov. 19, 2015.

(51) Int. Cl. A61K 31/4184; A61K 31/401

(52) U.S. Cl. CPC A61K 31/4184; A61K 31/401

(57) ABSTRACT

The present disclosure provides compositions and methods for treating multidrug-resistant nephrotic syndrome (MDR-NS) in pediatric subjects comprising atrasentan. Methods of reducing protenuria in a pediatric patient having MDR-NS are also provided.

### CROSS RELATED APPLICATIONS

This application claims priority to U.S. Provisional Applications No. 62/193,410 filed Jul. 16, 2015 and No. 62/257,536 filed Nov. 19, 2015, the contents of which are incorporated by reference in their entireties.

### BACKGROUND OF THE INVENTION

### Kidney Disease in Children

The high level group term (HLGT) of Nephropathy in the Medical Dictionary for Regulatory Activities (MedDRA) includes the MedDRA Preferred Term (PT): nephrotic syndrome. The reported incidence of childhood nephrotic syndrome (NS) in four studies was 1.15 to 2.3 per 100,000 [ISKDC 1981].

Initial treatment of NS is typically corticosteroids, and the majority of children respond to this treatment with a resolution of proteinuria, indicating complete remission. Overall, 10% to 20% of children with NS will not respond to initial steroid treatment, and other children will develop late resistance. These children will typically be treated with drugs, such as immunosuppressives and RAS inhibitors (ACEi or ARBs). Children who fail to achieve remission with those treatments, or in whom those treatments are judged to be futile due to known genetic or familial variation, are considered to have MDR (multi-drug resistant)-NS. Due to the rarity of this target patient population (MDR-NS), the true incidence and prevalence are currently unknown.

Although the population of patients with MDR-NS is small, no definitive treatments are currently available for them.

### Atrasentan

Atrasentan is an endothelin (ET) receptor type A (ETA) antagonist that effectively and selectively inhibits ET binding to the ETA receptor. Atrasentan decreases the binding affinity of ET without affecting the receptor density. The systematic (IUPAC) name for atrasentan is (2R,3R,4S)-4-(1,3-Benzodioxol-5-yl)-1-[2-(dibutylamino)-2-oxoethyl]-2-(4-methoxyphenyl)pyrrolidine-3-carboxylic acid.

Atrasentan belongs to the “-entan” class of compounds, which block the ETA and/or ETB receptors. ET is a 21-amino acid peptide, which is well known as the most potent endogenous vasoconstrictor. ET is classified into 3 types: ET-1, ET-2, and ET-3 [Kohan 2011]. In kidneys, both ET-1 and ET-3 show widespread tissue distribution. From a physiological perspective, ETs regulate renal blood flow, glomerular filtration, and sodium and water reabsorption. It has been demonstrated that ET-1 mRNA and renal ET-1 clearance are increased in association with proteinuria in kidneys of diabetic rats. ET-1 effects are exerted through two different receptors: ETA receptors localized to vascular smooth muscle cells and fibroblasts and ETB receptors predominantly localized to endothelial cells, and, to a lesser extent, vascular smooth muscle cells. ETB receptors located in the collecting duct of the kidney function to modulate sodium reabsorption and excretion in response to circulating ET-1 levels.

There is strong evidence that ET-1, a peptide with growth-promoting and vasoconstricting properties, has a central role in the pathogenesis of proteinuria via several different mechanisms (i.e., albuminuria), which is mediated via activation of the ETA receptor. Nonclinical studies have demonstrated that ET receptor antagonists can reverse proteinuric renal disease, and preliminary studies in humans with diabetic as well as nondiabetic renal disease have shown that these drugs have remarkable antiproteinuric effects, which are additive to those of standard antiproteinuric therapy. The atrasentan relative selectivity for ETA receptor over ETB receptor is greater than 1,800-fold.

Three other ET antagonists, bosentan, macitentan, and ambrisentan, are currently approved by both the European Medicines Agency (EMA) and the United States Food and Drug Administration (FDA) for the treatment of patients with pulmonary arterial hypertension. These compounds have been shown to be effective in improving exercise capacity and pulmonary symptoms related to interstitial lung scarring.

Currently, atrasentan is being developed for treatment of diabetic nephropathy (DN) in the adult patient population. While DN and MDR-NS are both characterized by podocyte dysfunction, DN generally does not exist in the pediatric population because the time course for onset of incipient DN varies from 10 to 15 years after the onset of diabetes.

Needed in the art is an improved method of treating pediatric MDR-NS.

### SUMMARY OF THE INVENTION

In one embodiment, the present invention is the use of atrasentan to treat multidrug-resistant nephrotic syndrome (MDR-NS) in pediatric subjects.

In one embodiment, one would administer to a pediatric subject, preferably 6 months until the age of puberty onset, a sufficient amount of atrasentan or a salt thereof, wherein the administration reduces, treats, improves or ameliorates the symptoms of MDR-NS.

In one embodiment, the present invention will reduce or delay or improve at least one of the following symptoms of nephrotic syndrome to where the symptom is not within the listed levels, which indicate the presence of NS:
Proteinuria >4 mg/m²/hour (or 50 mg/kg/day) or a urine protein-to-creatinine ratio >2.0 mg/mg
Hypoalbuminemia <2.5 g/dL
Presence of edema and hyperlipidemia.

In one embodiment, the treatment is for a period of at least 3 months.

In another embodiment of the invention, the pediatric subject has not responded, or responded poorly, to first-line steroid treatment or second-line drug treatments.

In another embodiment of the invention, the pediatric subject has not responded, or responded poorly, to first-line steroid treatment or is not considered suitable for second-line treatments.

In another embodiment of the invention, the method of administration is selected from the group consisting of oral, rectal, parenteral, intracisternal, intravaginal, intraperitoneal, topical, bucal administration and an oral or nasal spray.

In another embodiment of the invention, the disclosure provides a method of treating MDR-NS, comprising the step of administering to a pediatric subject an effective amount of atrasentan or a salt thereof and an angiotensin-converting-enzyme inhibitors (ACEi) or angiotensin II receptor blockers (ARBs), wherein the administration reduces, treats, improves or ameliorates at least one symptom of MDR-NS.

In some embodiments, the administration reduces the level of proteinuria in the patient by at least 10%, and in some embodiments at least 20%. In some embodiments, the method reduces the level of proteinuria below 4 mg/m²/hour (or 50 mg/kg/day).

### DESCRIPTION OF THE DRAWINGS

The patent or application file contains at least one drawing in color. Copies of this patent application publication with color drawings will be provided by the Office upon request and payment of the necessary fee.

FIG. 1A-D are graphs illustrating the effect of atrasentan on proteinuria (FIG. 1A) [mg/g of body weight] and mean arterial blood pressure (FIG. 1C) with prophylactic treatment. Untreated n=17, atrasentan 2.5 mg/kg/d n=3, atrasentan 5 mg/kg/d n=7, atrasentan 15 mg/kg/d n=8. FIG. 1B illustrates the effect of atrasentan on proteinuria with delayed treatment and FIG. 1D is a comparison of relative proteinuria in relation to week 5. Untreated n=21, atrasentan delayed 5 mg/kg/d n=9.

FIG. 2 is a graph of mean arterial blood pressure. Healthy n=11, untreated n=20, atrasentan 2.5 mg/kg/d n=3, atrasentan 5 mg/kg/d n=7, atrasentan 15 mg/kg/d n=8, atrasentan delayed 5 mg/kg/d n=9.

FIGS. 3A, 3B, 3C, 3D and 3E are graphs of biochemical parameters (creatinine=FIG. 3A, urea=FIG. 3B, serum albumin=FIG. 3C, cholesterol level=FIG. 3D) after atrasentan treatment. Healthy n=8, untreated week 4 n=14, untreated week 8 n=9, atrasentan 2.5 mg/kg/d n=3, atrasentan 5 mg/kg/d n=7, atrasentan 15 mg/kg/d n=8, atrasentan delayed 5 mg/kg/d n=9.

FIG. 4 is a graph of endothelin-1 gene expression. Week 2: healthy/untreated n=4/5; week 4: healthy/untreated n=5/5; week 6: healthy/untreated n=4/4; week 8: healthy/untreated n=4/3.

FIGS. 5 A, B and C are graphs of histopathology measurements. FIG. 5A graphs measurement of tubulointerstitial fibrosis, FIG. 5 B graphs measurement of glomerular schlerosis index, and FIG. 5 C graphs the average number of podocytes per glomerulus.

FIG. 6 is a graph of proteinuria levels over time (mg/g of body weight) for control (n=36-8), untreated (n=55-8), Atrasentan delayed (n=18).

FIG. 7 is a bar graph demonstrating the mean arterial blood pressure for control (n=11), untreated (n=11), Atrasentan wk 1 (n=5), wk2 (n=12), wk3 (n=6), week 4 (n=15), week 5 (n=11), week 6 (n=12, week 7 (n=7), week 8 (n=13), and week 9 (n=10).

FIG. 8 is a set of bar graphs depicting biochemical parameters measured.

FIGS. 9A-D are histolopathological results. FIG. 9A is a set of images of representative immunohistochemical staining. FIG. 9B is a graph showing podocyte number per glomeruium. FIG. 9C is a graph showing glomerular sclerosis index. FIG. 9C is a graph showing tubulointerstitial fibrosis (% effected area).

FIG. 10A-C are bar graphs showing expression levels of Endothelin-1 mRNA (10A), relative ET_{A}-R mRNA expression (10B), and relative ET_{B}-R mRNA expression.

FIG. 11 depicts Western Blot analysis of mRNA levels of ET-1.

FIG. 12A-C depicts podocin expression in Atrasentan treated animals compared to control and untreated. FIG. 12A shows protein expression via Western blot analysis. FIG. 12B shows Nphs2 mRNA expression as determined by qRT-PCR. FIG. 12C shows podocin expression as seen by immunofluorescence staining.

### DESCRIPTION OF THE INVENTION

The present invention is drawn to the treatment of MDR-NS with atrasentan. The first step of the method is to identify pediatric patients with multidrug-resistant nephrotic syndrome.

Diagnosis of MDR-NS and Identification of Patients

Diagnosis of MDR-NS involves identifying pediatric patients with primary nephrotic syndrome (i.e., not involving treatable systemic disease) who do not respond or respond poorly, to steroid or second line treatments, or are not considered suitable for second line treatments.

Nephrotic syndrome (NS) in children is traditionally classified as primary/idiopathic, which occurs in the absence of an identifiable cause, or secondary, which occurs in the presence of an identifiable causative process. The cardinal feature of nephrotic syndromes is the extensive leakage of plasma proteins into urine. There are five types of primary nephrotic syndromes:
Congenital nephrotic syndrome (CNS) of the Finnish type (CNF)
Diffuse mesangial sclerosis
Minimal change nephrotic syndrome (MCD)
Focal segmental glomerulosclerosis (FSGS)
Membranous glomerulopathy

In one embodiment, the pediatric nephrotic syndrome has been defined by:
Proteinuria >4 mg/m2/hour (or 50 mg/kg/day) or a urine protein-to-creatinine ratio >2.0 mg/mg
Hypoalbuminemia <2.5 g/dL
Presence of edema and hyperlipidemia.

Pediatric patients who have been diagnosed with NS will be treated by the present invention if they are multidrug resistant.

There is no known method of curative treatment for MDR-NS. Management of the disease state is directed at treatment to reduce proteinuria and prevent complications caused by NS and/or massive edema. The majority of children with NS will respond to first-line treatment with steroids. However, 10% to 20% of children with NS fail to respond to initial steroid treatment, and other children develop late resistance. Patients who do not respond to second-line treatments, or in whom second-line treatment is considered futile, are diagnosed with MDR-NS. Second-line treatments are treatments that are administered after a first line treatment does not work. Suitable second line treatments are known in the art and include, but are not limited to, alkylating agents, calcineurin inhibitors, levamisole, mycophenolate mofetil, or rituximab, among others. Second-line treatments are described in Andolino T P, Reid-Adam J. Nephrotic syndrome. Pediatr Rev 2015; 36:117-25, incorporated by reference in its entirety.

While not absolute, MDR-NS in many cases is associated with mutations in genes encoding for structural or regulatory proteins of the kidney filtration barrier located in the glomerular capillary wall. Nephrin Gene Mutations (Nephrotic Syndrome Type 1): Congenital nephrotic syndrome of the Finnish type, the most prevalent type of congenital nephrotic syndrome, is a recessively inherited disorder caused by mutations in the nephritic syndrome type 1 (NPHS1) gene encoding a major podocyte slit-diaphragm protein, nephrin. The syndrome is characterized by massive proteinuria detectable at birth, marked edema, and histologically characteristic radial dilatations of the proximal tubules. These histologically characteristic lesions are detected more frequently after 3 months of age, although they have also been identified in fetuses. Proteinuria in CNF patients starts in utero and can be detected in the first urine sample after birth. Due to the complete absence of nephrin, the resultant phenotype is typically severe with multiple complications and an unstable clinical course. As noted above, although CNF patients are likely to be resistant to steroids and other medical therapies, this patient population is not considered to be the most likely to benefit from atrasentan treatment. This is because of the lack of anticipated clinically meaningful benefit based on the mechanism of action of atrasentan and the underlying pathology.

Mutations in the nephrotic syndrome type 2 (NPHS2) gene, encoding for the podocyte protein, podocin, are commonly associated with SRNS in the pediatric population; in addition, these mutations may play an important role in the development of CNS. The NPHS2 gene mutations were reported to account for half of the CNS cases involving 80 families in Europe, while mutations in the NPHS1 gene were responsible for only one-third of CNS cases. The NPHS2 mutations have also been found in the Japanese patients with CNS and those from other regions. These mutations are typically classified as severe, leading to nonfunctional podocin protein, which is often truncated. Because podocin is a podocyte adapter protein involved in proper targeting of nephrin into slit diaphragm, nephrin expression may also be compromised in children with nephrotic syndromes associated with NPHS2 mutations. NPHS2 mutations causing renal disorders are highly heterogeneous, but almost all are resistant to corticosteroid therapy. The most common presenting feature of nephrotic syndrome in pediatric patients is persistent proteinuria (>50 mg/kg/day), usually manifesting in patients younger than 3 years of age.

Because NHPS2 nephrotic syndrome typically does not respond to immunosuppressive therapy, it is encompassed by the term steroid-resistance nephrotic syndrome (or SRNS) described above. The severity of proteinuria and other clinical findings in patients with NPHS2 mutations vary to a greater extent than those in patients with NPHS1 mutations. The renal histology in patients with NPHS2 mutations typically reveals focal and segmental glomerular sclerosis or minimal-change disease. These patients usually develop ESRD in early childhood, although in some children the decline in renal function is slower and ESRD develops in adolescence. While SRNS by definition does not respond to steroid therapy, patients with SRNS associated with NPHS2 mutations have been shown to have a lower rate of recurrence of disease after renal transplantation. In a mutational analysis of 8 NPHS2 exons in 190 patients from 165 families with SRNS and 124 patients from 120 families with SSNS, homozygous or compound heterozygous mutations of NPHS2 were observed in approximately one-quarter of families with SRNS compared with none of the families with SSNS.

Several research groups have shown that the patients with SRNS who have multiple mutations have a younger age at disease onset than those who have no mutations. In one study of 319 patients with SRNS, the mean age at disease onset of the patients without 2 NPHS2 mutations was 108.3 months (9.0 years) in those with sporadic SRNS and 69.9 months (5.8 years) in those with autosomal recessive SRNS compared with a mean age at onset of 41.2 months (3.4 years) for those with 2 NPHS2 mutations (P<0.001 and P=0.019, respectively) [Weber 2004]. In another study of 430 patients with SRNS of whom 267 had no mutation and 78 had multiple mutations, the mean age at onset was 6.4 years for those with no mutation and 2.61 years for those with multiple mutations (P<0.0001).

Based on available epidemiology data, it is expected that a significant number of the intended target patient population may have mutations in the NPHS2 gene.

Mutations in Wilms' tumor suppressor gene (WT1), which plays a key role in the embryonic development of the kidney and genitalia, account for a small percentage of CNS cases. Patients with WT1 mutations may experience moderate proteinuria, while kidney biopsy typically shows diffuse mesangial sclerosis of glomeruli. Mutations in the laminin-beta 2 gene playing a crucial role in the network structure and anchoring of the glomerular basement membrane to podocyte foot processes are also linked to development of CNS.

Another genetic disorder associated with CNS is Galloway-Mowat syndrome, characterized by nephropathy accompanied by central nervous system anomalies, including microcephaly, psychomotor retardation, and brain abnormalities. In addition to Galloway-Mowat syndrome, other unique combinations of CNS and extrarenal defects are reported, including mitochondrial cytopathy, nail-patella syndrome, congenital disorder of glycosylation type I, Herlitz junctional epidermolysis bullosa, and mutations in the phospholipase C epsilon gene.

One skilled in the art would understand that the diagnosis of MDR-NS is purely clinical, so patients eligible for the treatment of the present invention are not limited to any of the above-mentioned mutations. In one embodiment, patients for the present invention have at least one mutation in any of these genes. In one embodiment, patients for the present do not have any of the mutations.

Nephrotic syndrome may be the result of other pathologies and may not typically be as suitable for atrasentan treatment, although we envision that in some cases, atrasentan treatment will be useful. Some infections may also cause congenital and infantile nephrotic syndromes, especially in developing countries. These include syphilis, toxoplasmosis, congenital rubella, hepatitis B, cytomegalovirus, and human immunodeficiency virus. First-line therapy for CNS arising from infections is aimed at the infectious organism. For this reason, nephrotic syndromes arising from infectious etiologies will not typically be treated with atrasentan before resolution of the underlying pathology. Other secondary forms of CNS include maternal systemic lupus erythematosus and neonatal alloimmunization against neutral endopeptidase present on podocytes. Treatment of these disease states is directed at the primary disease process, making patients with these conditions typically treated with other drugs before atrasentan treatment.

Pediatric patients with MDR-NS, while having symptom similarities with DN, have a much shorter time course for onset as compared to adults, as the onset of incipient DN varies from 10 to 15 years after the onset of diabetes in adult populations. Thus, pediatric patients with MDR-NS have a different course of symptoms and disease progression than adult patients.

Treatment of the Present Invention

In one embodiment of the invention, a pediatric patient will receive atrasentan treatment, typically via administration as described in other atrasentan treatment literature or as described below. The treatment is expected to reduce or delay one or more MDR-NS symptoms.

The typical target pediatric patient population for the method of the present invention is MDR-NS patients between 6 months and the onset of puberty who present with persistent or refractory nephrotic syndrome that has failed to respond to steroids and other appropriate therapeutic alternatives, although patients in a wider variety of ages will benefit. This population also encompasses patients with SRNS.

The onset of puberty varies among individuals; however, puberty usually occurs in girls between the ages of 10 and 14, while in boys it generally occurs later, between the ages of 12 and 16. For a particular patient, the timing of his or her onset of puberty can be determined by on skilled in the art using a number of factors known in the art.

In one embodiment, the target pediatric patient population of the method of the present invention is MDR-NS patients 6 months to the onset of puberty, or to 16 years of age, or to 14 years of age, or to 12 years of age, or to 10 years of age, or preferably to 8 years of age. In one specific embodiment, the target pediatric patient for the method of the present invention is between 6 months to 8 years of age.

In one embodiment, a pediatric patient for the treatment of the present invention is 6 months to 16 years of age. In one embodiment, a pediatric patient for the treatment of the present invention is 6 months to 14 years of age. In one embodiment, a pediatric patient for the treatment of the present invention is 6 months to 12 years of age. In one embodiment, a pediatric patient for the treatment of the present invention is 6 months to 10 years of age. In one embodiment, a pediatric patient for the treatment of the present invention is 6 months to 8 years of age.

The treatment regimen of the present invention would typically occur as add-on therapy in patients who have failed first-line or other conventional therapy. In one embodiment of the invention, the pediatric subject has not responded, or responded poorly, to first-line steroid treatment or second-line drug treatments. In one embodiment of the invention, the pediatric subject has not responded, or responded poorly, to first-line steroid treatment or is not considered suitable for second-line treatments.

In one embodiment, the treatment is for a period of at least 3 months.

In one embodiment, the treatment is for a period of at least 6 months.

Albuminuria/proteinuria assessment, preferably by at least 6 months post treatment initiation, is a typical symptom guideline for evaluation of clinical response of the treatment of the present invention. In one embodiment, a treatment will modify the patient's albuminuria/proteinuria profile by at least 10%, in some embodiments by at least 20%, and in some further embodiments by at least 30%. In another embodiment, the modification will be so that symptom levels are below those listed above as indicative of renal disease. For example, the patient's proteinuria measurement will be less than 4 mg/m²/hour (or 50 mg/kg/day) or a urine protein-to-creatinine ratio less than 2.0 mg/mg or the patient's hypoalbuminemia will be greater than 2.5 g/dL. In some embodiments, the reduction or delay of one or more symptoms of MDR-NS is demonstrated by a patient having hypoalbuminemia by the increase in the level of albumin in the blood. The level of albumin in the patient may be increased by at least 10%, alternatively at least 20%, alternatively at least 30%. In some embodiments, the reduction or delay of one or more symptoms of MSD-NS is the reduction of edema and/or hyperlipidemia in the patient. Not to be bound by any theory, patients improved in the disease state will have a reduced protein loss, which in turn may improve serum albumin levels.

In some embodiments of the invention, the treatment of a pediatric patient with atrasentan in an effective amount to delay the onset or progression of end-stage renal disease (ESRD).

In some embodiments, the reduction or delay of one or more symptoms of MSD-NS is demonstrated by the reduction of glomerular hyperfiltration or reducing in tubulointerstitial inflammation.

Our results show that the atrasentan treatment is most successful after the disease state has been established. In one embodiment of the invention, one would evaluate the patient and determine that MDR-NS is fully established. In one embodiment, persistent proteinuria is an indication that MDR-NS is established. In some instances, one of methods that can be used to assess the establishment of MDR-NS is to measure development of FSGS related to the upregulation of endothelin.

In some embodiments, the treatment with atrasentan will be combined with one or more second-line drug therapy. Suitable second-line drug therapy include, but are not limited to, for example, immunosuppressives and Renin-angiotensin system (RAS) inhibitors (ACEi or ARBs). Immunosuppresives include, but are not limited to, for example, corticosteroids (e.g., prednisone, dexamethasone, etc.), calcineurin inhibitor (e.g., cyclosporine, tacrolimus), alkylating agent (e.g., cyclophosphamide, chlorambucil, etc.) mycophenolate mofeitil (MMF, T and B-cell proliferation inhibitor), rituximab (monoclonal antibody specific to CD20 found on B cells) and the like.

Suitable RAS inhibitors include, but are not limited to, for example, angiotensin-converting-enzyme inhibitors (ACEi) or angiotensin II receptor blockers (ARBs). Suitable ACEi therapies include, but are not limited to, for example, alacepril, benzapril, captopril, ceronapril, cilazapril, delapril, enalapril, enalaprilat, eosinopril, fosinopril, imidapril, lisinopril, moexipril, moveltipril, omapatrilat, perindopril, quinapril, ramipril, sampatrilat, spirapril, temocapril, trandolapril, and combinations thereof, among others. Suitable ARBs include, but are not limited to, for example, candesartan, eprosartan, irbesartan, losartan, olmesartan, tasosartan, telmisartan, valsartan or combinations thereof, among others.

In one embodiment, the present treatment comprises treating with Atrasentan and an ACEi inhibitor in an effective amount to reduce at least one symptom of MDR-NS. In some embodiments, the combination has a synergistic effect, e.g., provides an increased reduction of at least one symptom as compared to either drug alone. In one embodiment, the treatment comprises administering an effective amount of Atrasentan and Candesartan.

### Pharmaceutical Compositions

When employed as a pharmaceutical, a compound of the invention is typically administered in the form of an oral pharmaceutical composition. Other suitable forms of administration are discussed below. Atrasentan, solvates thereof, crystalline forms thereof, salts thereof, or any other suitable clinical dose forms thereof, can be made by synthetic chemical processes by one skilled in the art, for example, as described in U.S. patent application Ser. Nos. 09/714,934, 10/266,270, 11/063,476, 12/037,510, 08/458,094, 08/457,215, 08/457,063, 08/457,935, 08/457,331, 08/457,414, 08/457,418, 09/572,493, 11/502,798, 08/293,349, 08/334,717, 08/442,575, 08/497,998, 08/600,625, 08/794,506, 08/905,913, 09/048,955, 09/634,661, 09/653,563, 11/229,892, 11/230,043, 11/229,894, 11/229,922, 14/324,603, 14/133,297 and 14/594,742.

Suitable compositions can be prepared in a manner well known in the pharmaceutical art and comprise a therapeutically effective amount of atrasentan or a pharmaceutically acceptable salt thereof together with a pharmaceutically acceptable carrier. The phrase “pharmaceutical composition” refers to a composition suitable for administration in medical or veterinary use.

The pharmaceutical compositions that comprise atrasentan, alone or in combination with further therapeutically active ingredient, may be administered to the subjects orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which may serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous diluents, solvents, or vehicles include water, ethanol, polyols (such as glycerol, propylene glycol, polyethylene glycol and the like), vegetable oils (such as olive oil), injectable organic esters (such as ethyl oleate), and suitable mixtures thereof. Proper fluidity may be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it may be desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle.

Injectable depot forms may be made by forming microencapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In certain embodiments, solid dosage forms may contain from 1% to 95% (w/w) of a compound of atrasentan. In certain embodiments, atrasentan or pharmaceutically acceptable salts thereof, may be present in the solid dosage form in a range of from 5% to 70% (w/w). In such solid dosage forms, the active compound may be mixed with at least one inert, pharmaceutically acceptable carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

The pharmaceutical composition may be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form may be a capsule, tablet, cachet, or lozenge itself, or it may be the appropriate number of any of these in packaged form. The quantity of active component in a unit dose preparation may be varied or adjusted from 0.1 mg to 1000 mg, from 1 mg to 100 mg, or from 1% to 95% (w/w) of a unit dose, according to the particular application and the potency of the active component. The composition may, if desired, also contain other compatible therapeutic agents.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician may evaluate factors such as the circulating plasma levels of the compound, compound toxicities, and/or the progression of the disease, etc.

For administration, compounds may be administered at a rate determined by factors that may include, but are not limited to, the LD₅₀ of the compound, the pharmacokinetic profile of the compound, contraindicated drugs, and the side-effects of the compound at various concentrations, as applied to the mass and overall health of the subject. Administration may be accomplished via single or divided doses.

The compounds utilized in the pharmaceutical method of the invention may be administered at the initial dosage of about 0.001 mg/kg to about 100 mg/kg daily. In certain embodiments, the daily dose range is from about 0.1 mg/kg to about 10 mg/kg. In certain embodiments, the daily dose range is from about 0.1 mg/kg to about 5 mg/kg. Suitable dosages may contain any amounts in-between, for example, but not limited to, about 0.1 mg/kg, about 0.25 mg/kg, about 0.5 mg/kg, about 0.75 mg/kg, about 1.0 mg/kg, about 1.25 mg/kg, about 1.5 mg/kg, about 1.75 mg/kg, about 2 mg/kg, about 5 mg/kg, about 10 mg/kg and so forth. For example, suitable dosages may comprise, 0.25 mg, 0.5 mg, 0.75 mg, 1.25 mg and 1.75 mg. The dosages, however, may be varied depending upon the requirements of the subject, the severity of the condition being treated, and the compound being employed. Determination of the proper dosage for a particular situation is within the skill of the practitioner. Treatment may be initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which may be prepared by mixing the compounds with suitable non-irritating carriers or carriers such as cocoa butter, polyethylene glycol, or a suppository wax which are solid at room temperature but liquid at body temperature and therefore melt in the rectum or vaginal cavity and release the active compound.

Compounds may also be administered in the form of liposomes. Liposomes generally may be derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes may be used. The present compositions in liposome form may contain, in addition to a compound of the invention, stabilizers, preservatives, excipients, and the like. Examples of lipids include, but are not limited to, natural and synthetic phospholipids, and phosphatidyl cholines (lecithins), used separately or together.

Methods to form liposomes have been described, see example, Prescott, Ed., Methods in Cell Biology, Volume XIV, Academic Press, New York, N.Y. (1976), p. 33 et seq.

Dosage forms for topical administration of a compound described herein include powders, sprays, ointments, and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

A compound of the invention may also be administered in sustained release forms or from sustained release drug delivery systems.

In one version of the present invention, one would combine the atrasentan treatment with a dose of at least one of an angiotensin converting enzyme inhibitor or an angiotensin II receptor blocker.

### EXAMPLES

### Example 1

We have analyzed results from studies in a transgenic mouse model where the mice were treated with atrasentan. Using the Cre recombinase technology, we generated a novel inducible mouse model of podocin-related nephrotic syndrome. Cross-breeding of animals carrying one foxed podocin construct, one podocin allele with the R140Q mutation, and tamoxifen-sensitive inducible Cre enabled us to induce hemizygosity for the R140Q mutant. In this work, we were able to examine the human missense mutation R138Q of NPHS2 gene identified in pediatric patients with nephrotic syndrome in vivo in an inducible mouse model of R140Q mutation (murine analogue to the human mutation). The induced animals with Nphs2^{R140Q/-} genotype developed proteinuria within the first week after the induction with tamoxifen, which reached its maximum at week 4 and 5. Podocytes injury in our model resulted in progressive renal disease, demonstrating phenotype of nephrotic syndrome in human, such as proteinuria, hyperlipidemia, hypertension and kidney failure. Nphs2^{R140Q/-} mice displayed clear difference in plasma level of cholesterol and albumin already two weeks after the induction. Furthermore, from the end of the week four of observation the values of plasma urea and creatinine started to increase slightly and the creatinine clearance appeared to reduce, pointing to the beginning of renal failure.

Seven animals were treated with 5 mg/kg/d atrasentan and another three animals were treated with the lower dose of 2.5 mg/kg/d atrasentan via food intake for four weeks. Furthermore, we started a new group of 8 animals with a higher dose treatment of 15 mg/kg/d atrasentan.

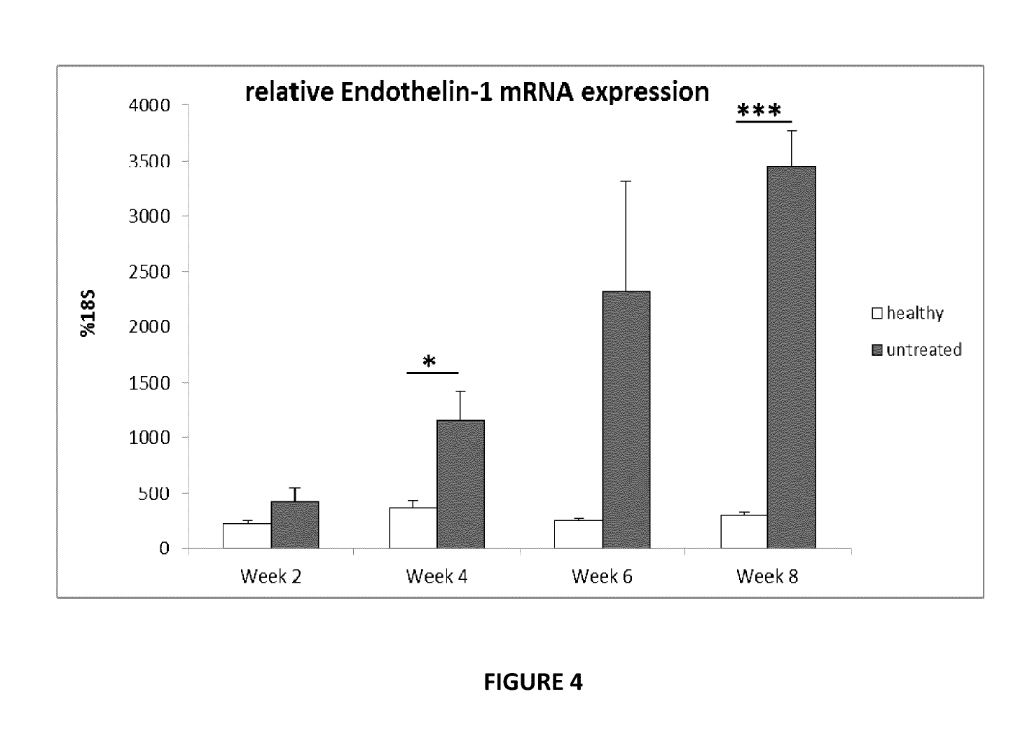

We found that there is a late upregulation of endothelin-1 mRNA expression (FIG. 4) in the course of disease, raising the possibility that atrasentan is only effective when FSGS (focal segmental glomerulosclerosis) is fully established. Therefore, we started a therapeutic treatment of 5 mg/kg/d with a four-week delay from when animals were shown to establish proteinuria (n=9).

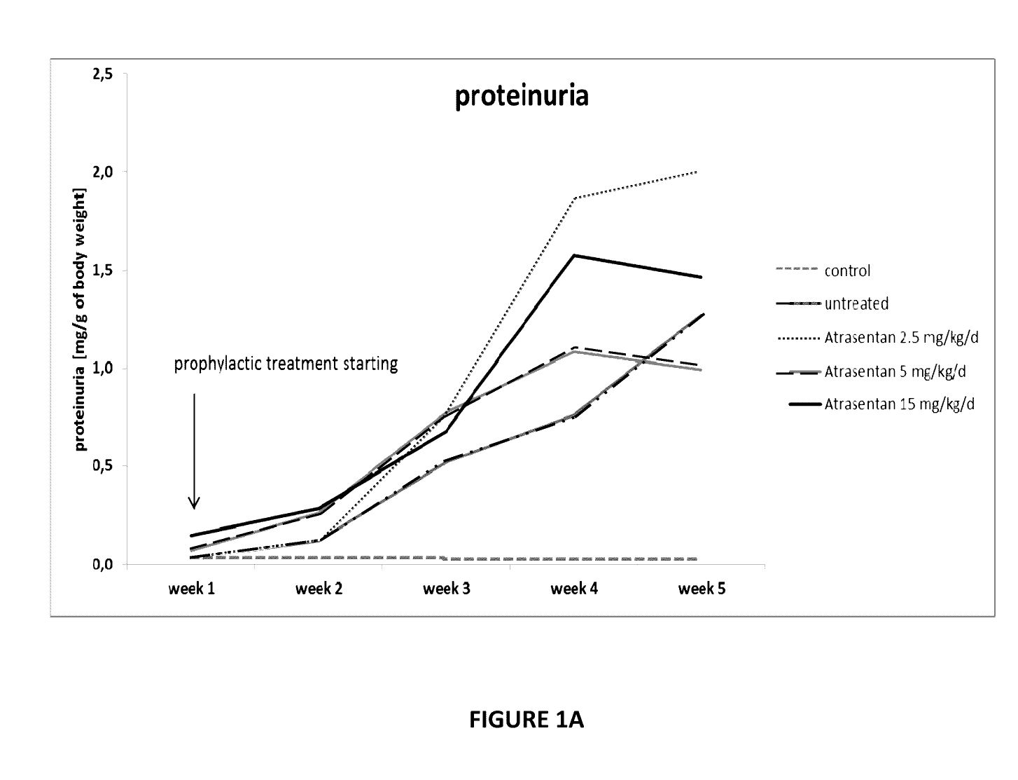

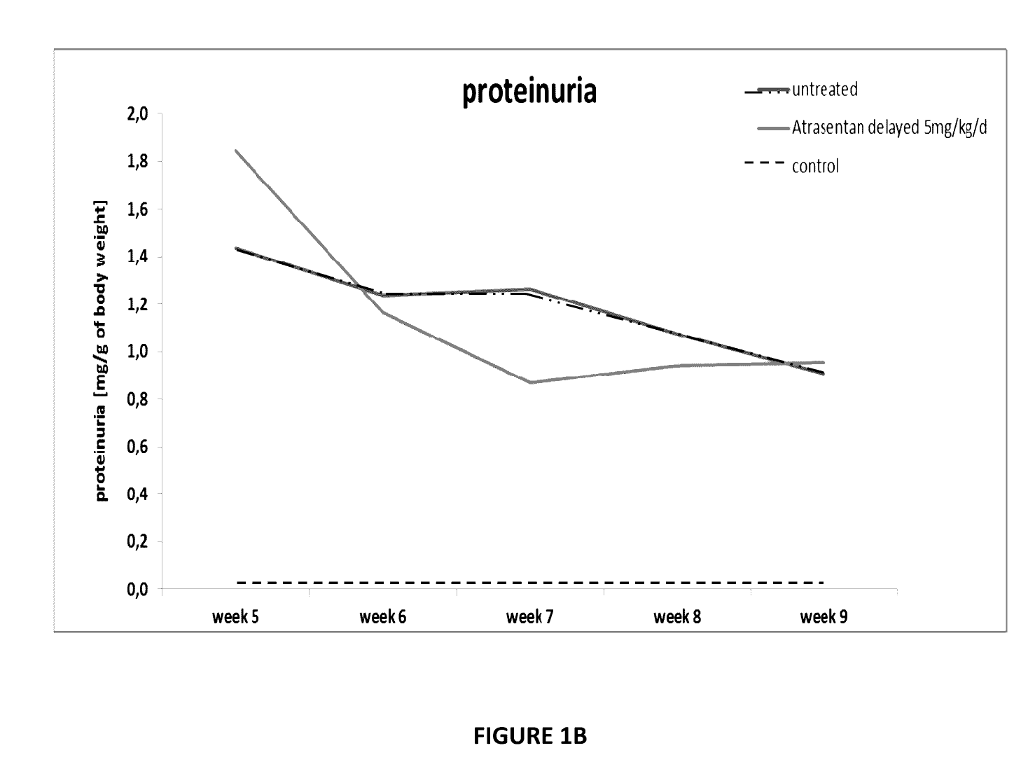

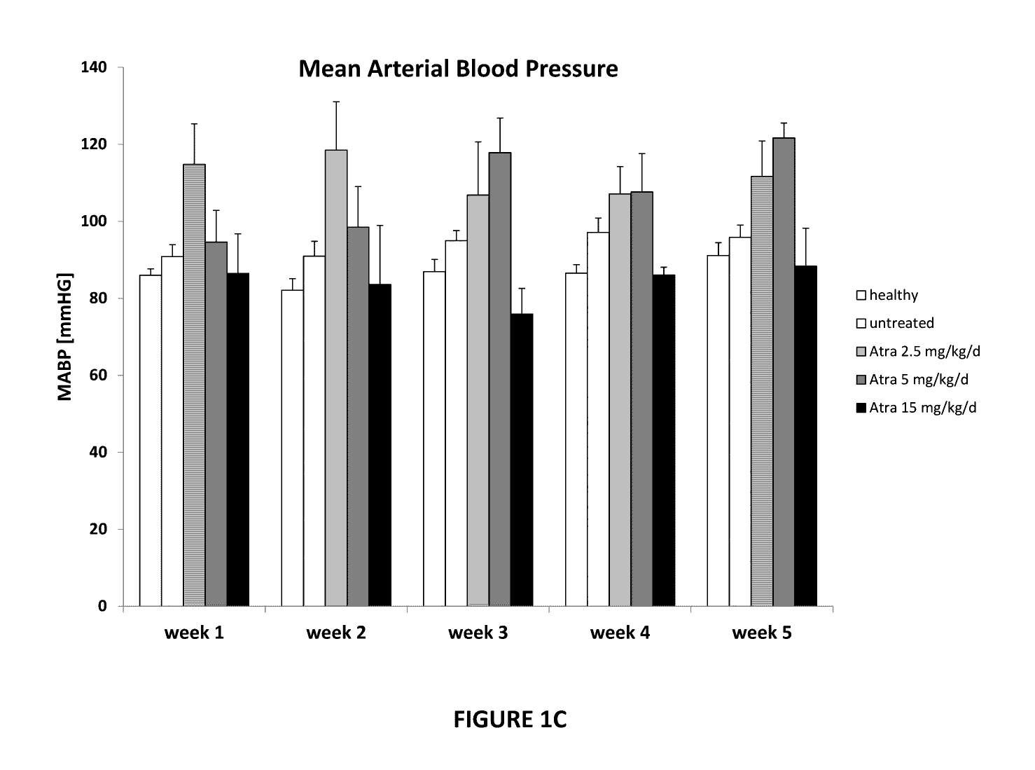

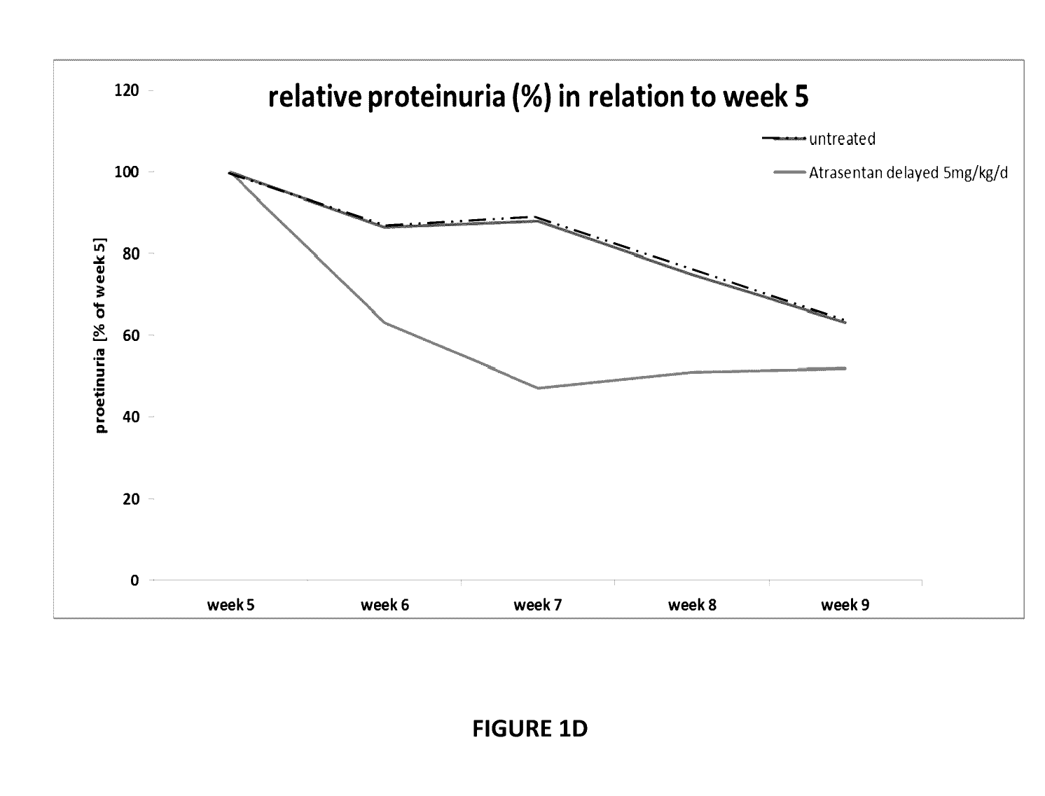

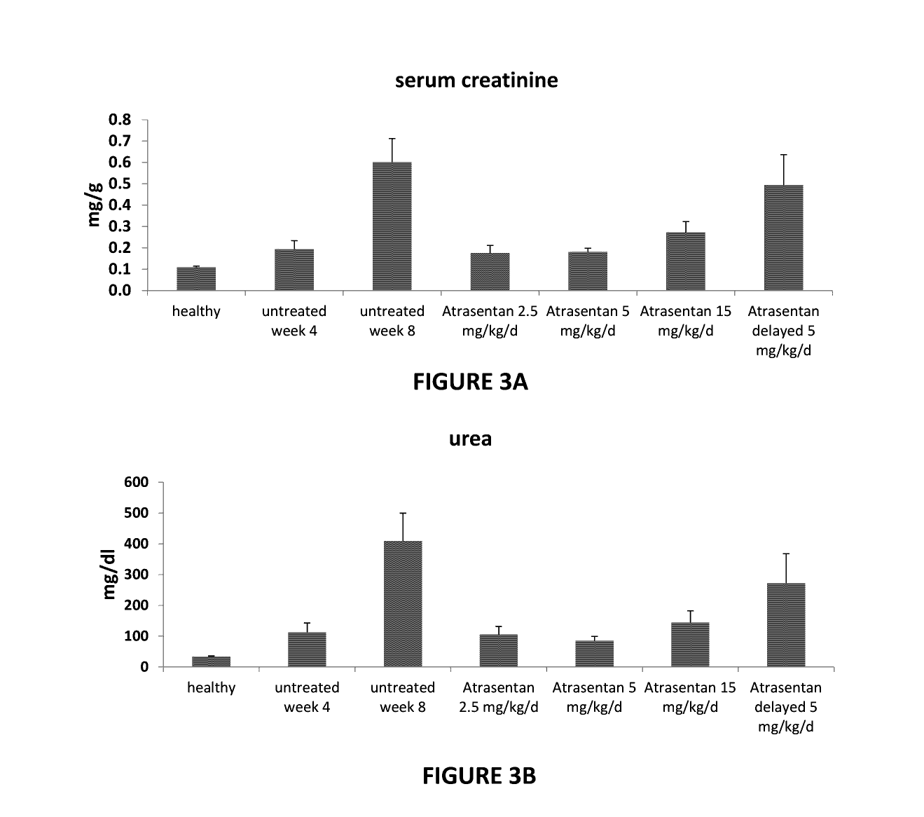

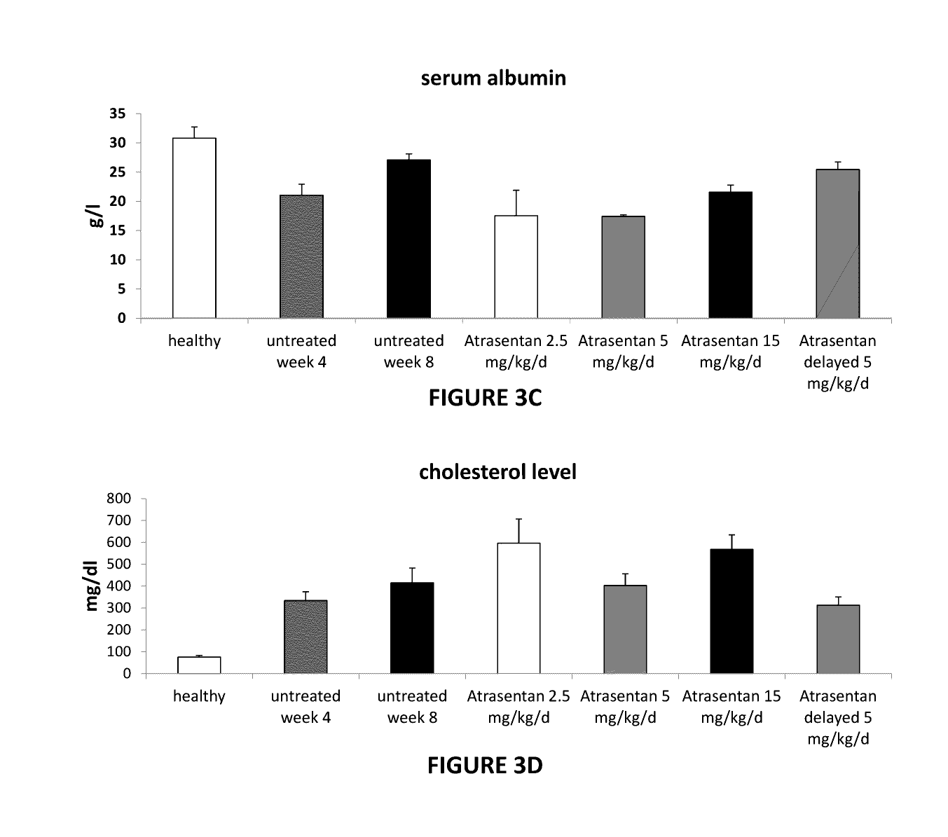

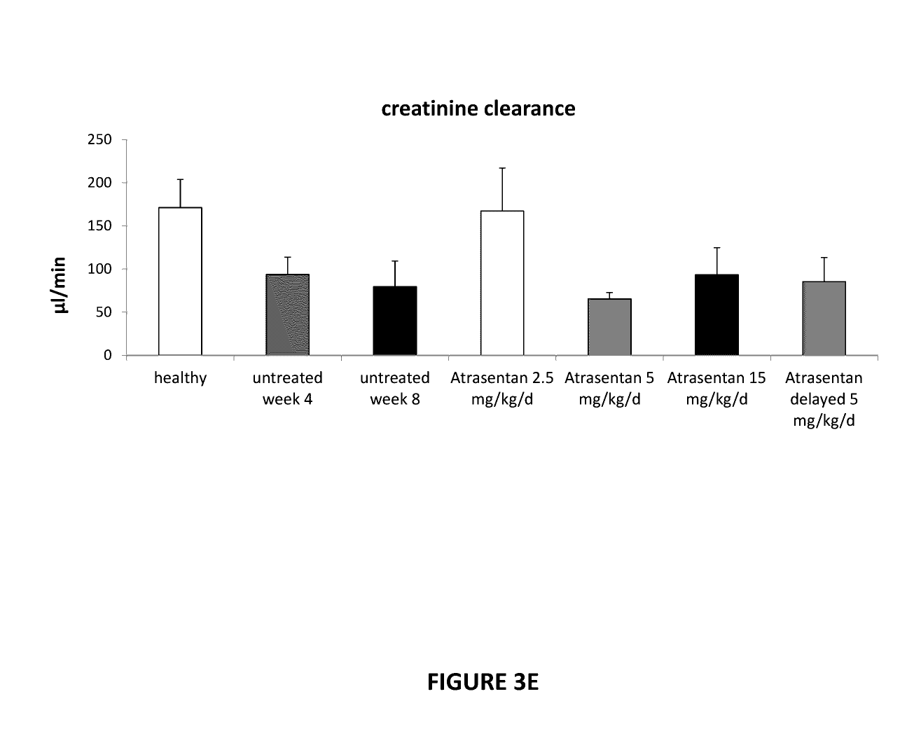

Proteinuria (FIG. 1A, FIG. 1B, and FIG. 1D), weight gain and blood pressure (FIG. 1C) were monitored once weekly. All animals were sacrificed at the end of the observation period at week 5 (or week 9 with delayed treatment) and the biochemical parameters (FIG. 3A-E) were determined. All results were compared to two control groups of healthy and sick untreated animals.

Referring to FIG. 1A-D, no beneficial effect in attenuating proteinuria was seen in animals with immediate or prophylactic treatment of atrasentan, regardless of the dose administered.

Referring to FIG. 1C, animals treated with 2.5 mg/kg/d and 5 mg/kg/d atrasentan demonstrated a higher blood pressure comparing to untreated and healthy animals. Animals treated with the higher dose of 15 mg/kg/d display a lower blood pressure compared to untreated animals.

Referring to FIGS. 1B and D, when we started atrasentan 5 mg/kg/d four weeks after induction, when proteinuria was fully established, an effective attenuation of proteinuria was observed. The experiment reported in FIG. 1B records n=21 untreated and n=9 atrasentan treated animals (delayed 5 mg/kg/d).

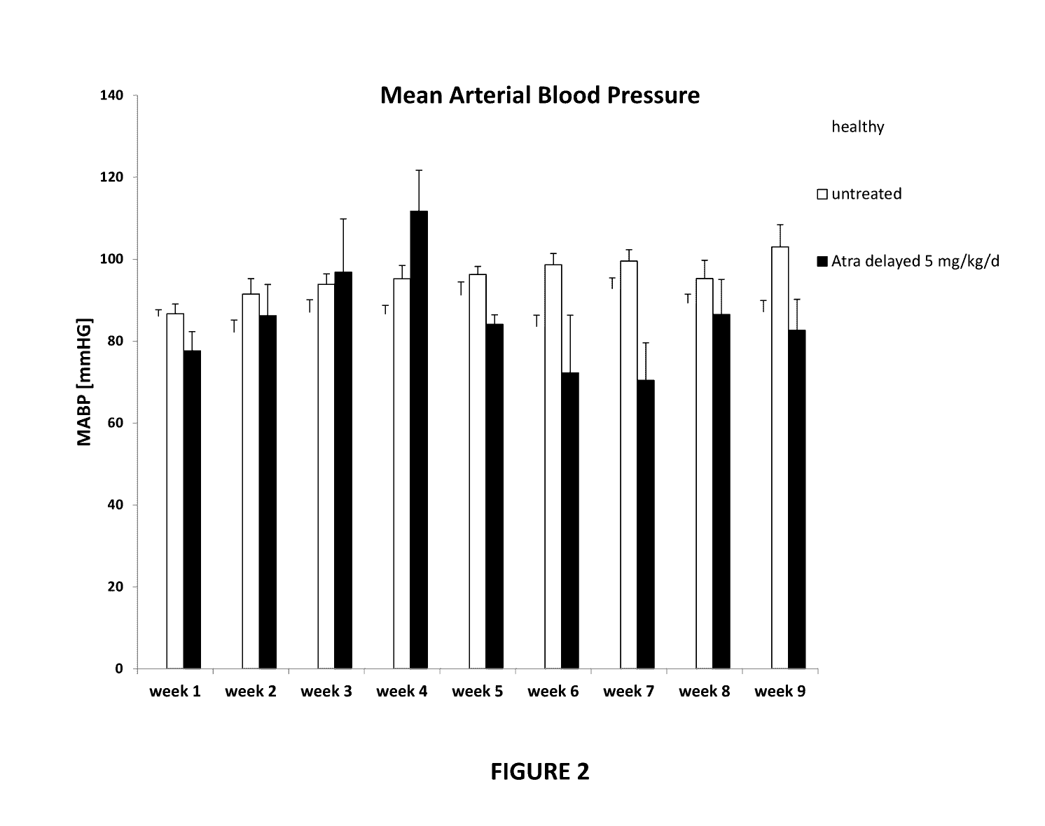

Referring to FIG. 2, delayed atrasentan treatment also lowered blood pressure compared to untreated animals.

FIGS. 3A-E record biochemical parameters of the treated animals.

FIG. 4 indicates that real-time rtPCR showed a late upregulation of endothelin-1 expression in the course of disease, reaching significance only by week 4.

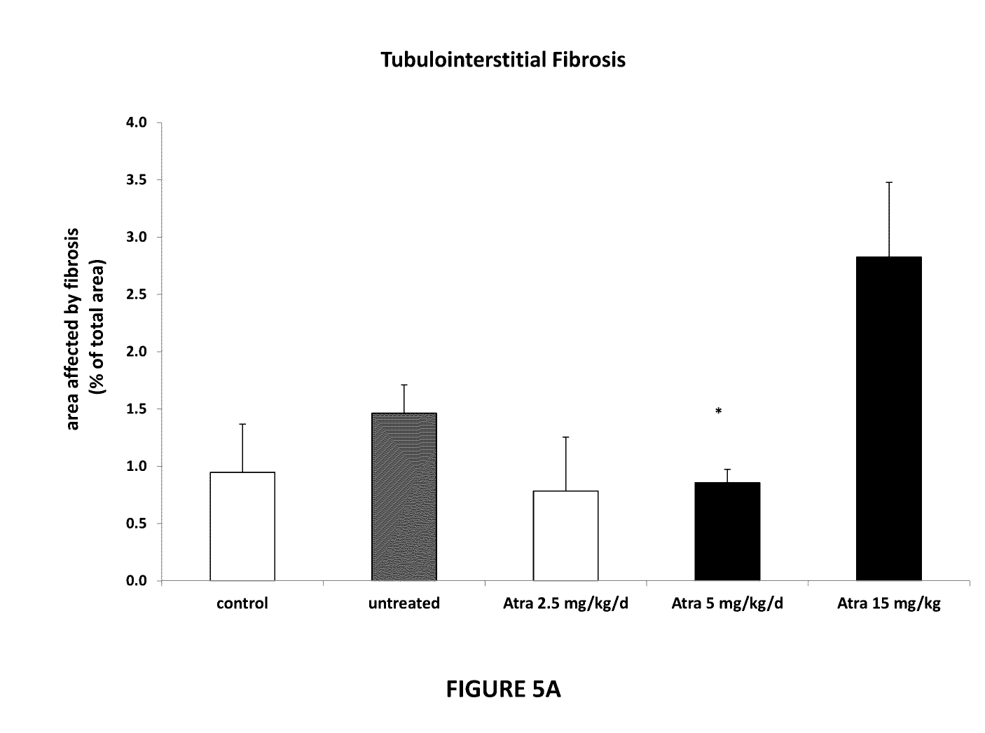

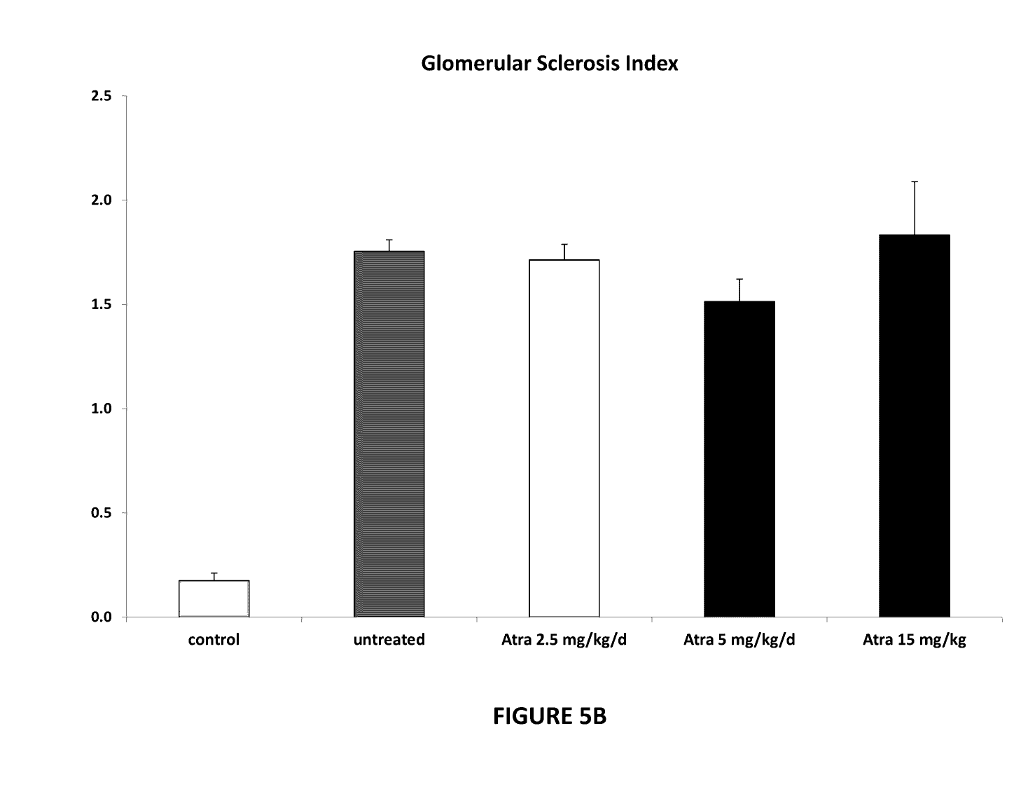

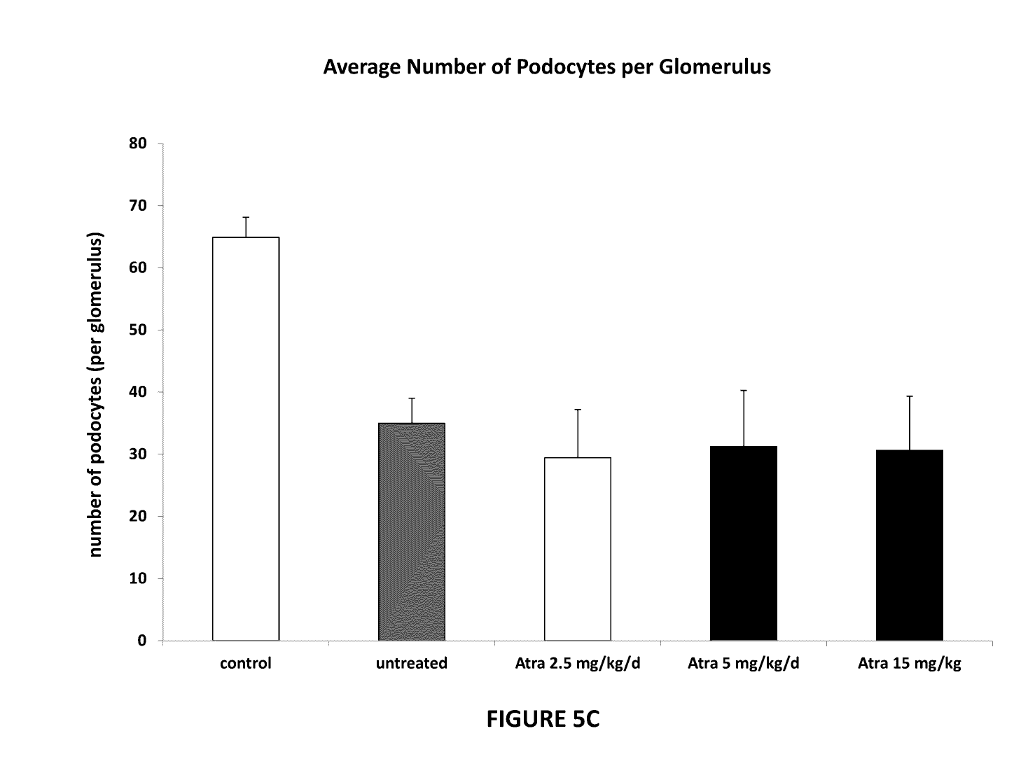

FIG. 5A shows that tubulointerstitial fibrosis determined by Sims Red staining. Results demonstrate a reduced area affected by fibrosis in treated animals with dose of 2.5 and 5 mg/kg/d Atrasentan (*p<0.05 compared to untreated group). Animals treated with 15 mg/kg/d showed an increase of tubulointerstial fibrosis.

FIG. 5B demonstrates sclerosis within the glomerular tuft examined by PAS staining of 3 μm paraffin sections. Quantification was done with Image ProPremier (glomerular sclerosis index—GSI, 50 glomeruli/animal). No improvement was seen in GSI in all treatment groups.

FIG. 5C tabulates podocyte numbers per glomerulusWT1-via immunostaining of glomerular cross-sections (4 μm and 10 μm). Quantification was done with ImageJ according to Animal Models of Diabetic Complications Consortium protocol. 50 glomeruli/animal. We found no effect on the number of podocytes per glomerulus in treated animals.

In summary, we saw no antiproteinuric effect of atrasentan in the first 4 weeks after induction of the podocin defect, regardless of the dose applied—much different from ACE/ARBs, which are strongly antiproteinuric.

We then checked ET1 expression in the kidney over time and found that the ET1 system is gradually up-regulated only after a few weeks of active disease.

We then proceeded to a second series, starting atrasentan at 4 weeks when FSGS is fully established and ET1 expression is up. (See FIG. 1B) With this design, we were excited to see an antiproteinuric effect attributable to atrasentan treatment.

### Example 2

### Delayed Treatment

Using the same mouse model as Example 1, we found that there is a late upregulation of endothelin-1 mRNA expression (FIG. 4) in the course of disease, raising the possibility that Atrasentan is only effective when FSGS is fully established. Therefore, we started the treatment after 4 weeks of disease state (5 mg/kg/d) when animals are showing high proteinuria, performing 4-weeks treatment duration (n=14) and 8 weeks treatment duration (n=4), respectively.

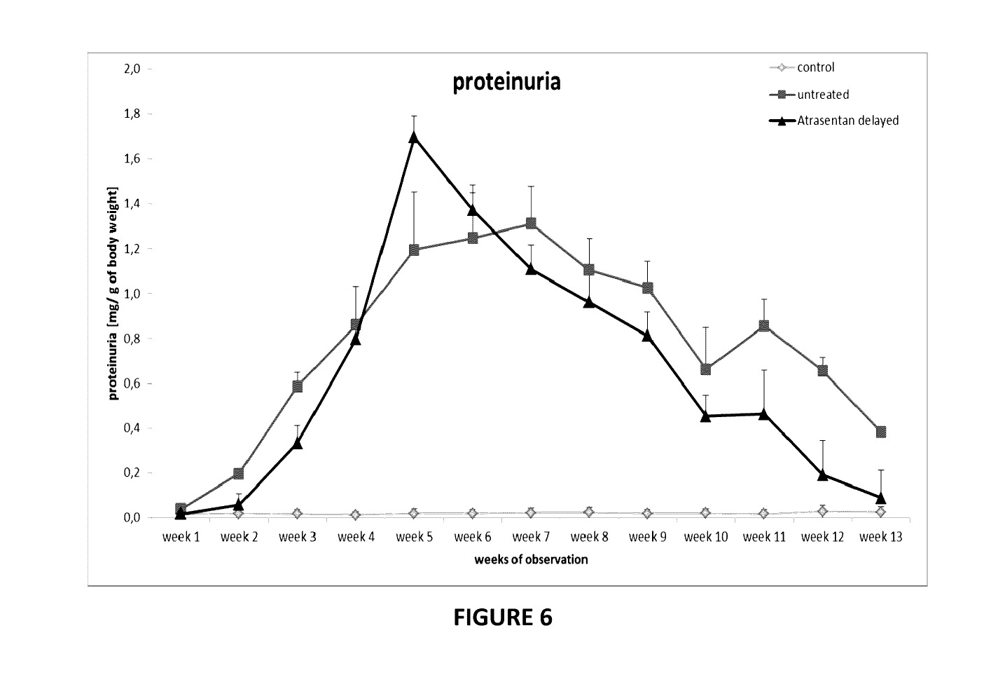

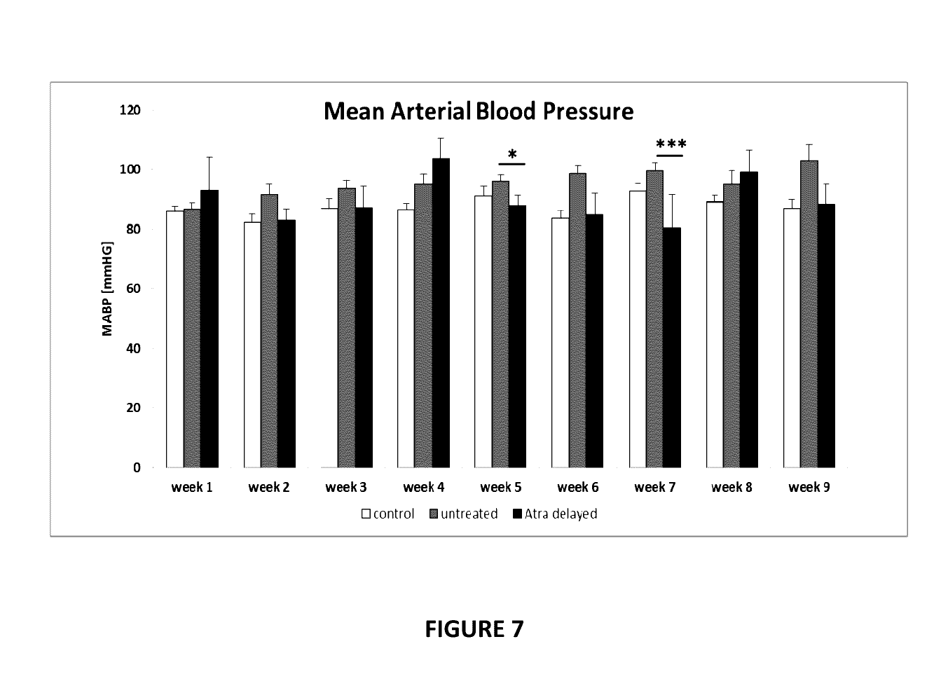

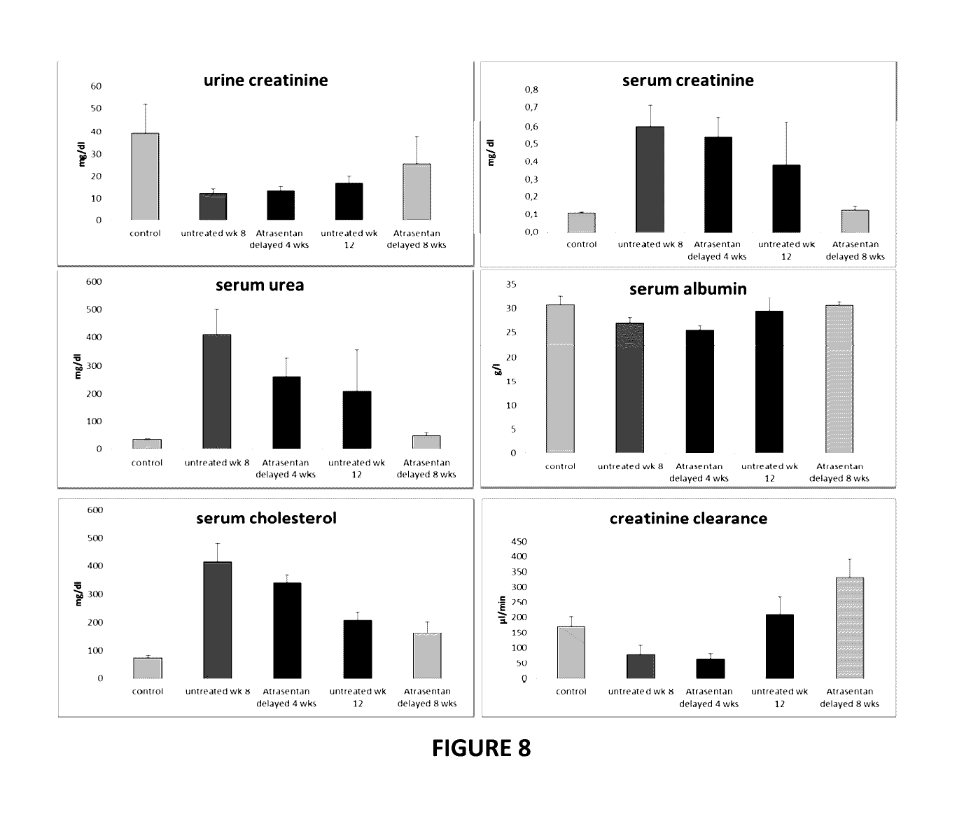

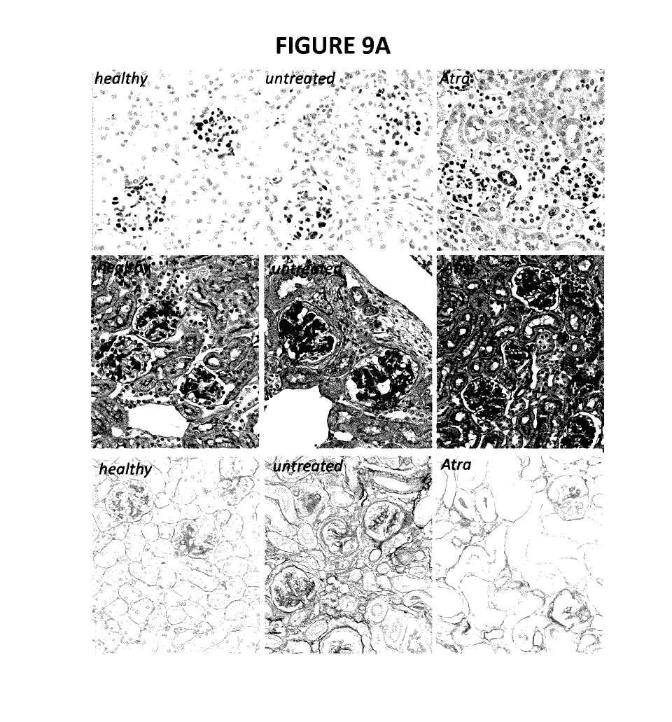

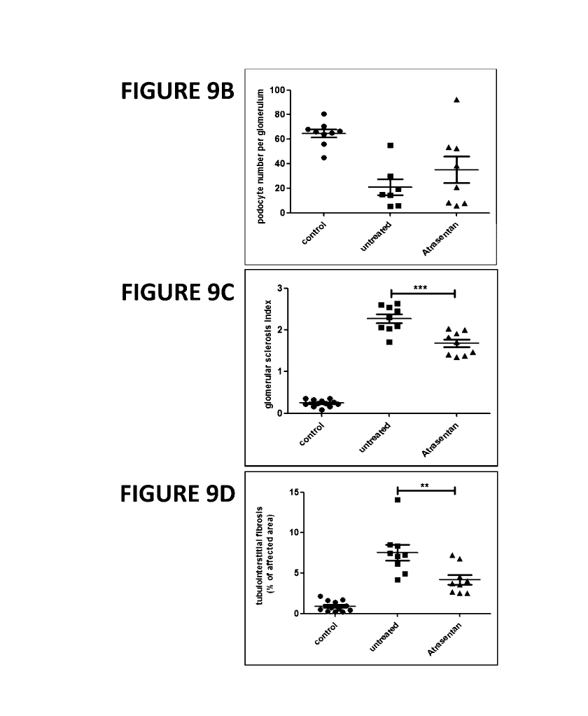

Proteinuria (FIG. 6), weight gain and blood pressure (FIG. 7) were monitored at least every 2 weeks. All animals were sacrificed at the end of the observation period and biochemical parameters (FIG. 8) as well as histological changes (FIG. 9) were determined consequently. All results were compared to control groups of healthy and sick untreated animals.

FIG. 6 demonstrates that after drug administration, proteinuria reaches a peak and declines afterwards progressively. FIG. 7 demonstrates that animals treated with four-week delay with doses of 5 mg/kg/d Atrasentan showed a significant decrease of blood pressure compared to untreated animals at week 5 and 7 (***p=0.001, *p=0.047)

FIG. 8 shows results of biochemical parameters with control (healthy mice, n=8), untreated week 8 (n=9), Atrasentan delayed 4 weeks (n=14), untreated week 12 (n=6) and Atrasentan delayed treatment 8 weeks (n=3) (Atrasentan delayed are mice who begin Atrasentan treatment after four weeks of disease state). FIG. 9A are exemplary images of healthy, untreated and Atrasentan treated kidney samples. FIG. 9B-D shows the results from histological examination from 9 out of 14 delayed treated animals (4 weeks of treatment). FIG. 9B shows podocyte number per glomeruium, FIG. 9C shows glomerular sclerosis index per animal, and FIG. 9D shows tubulointerstitial fibrosis (% affected area). Histopathological examinations demonstrated that glomerulosclerosis and tubulointerstitial fibrosis are mitigated in the Atrasentan treated animals as compared to sick untreated animals of the same age. No notable effects were seen on podocyte population.

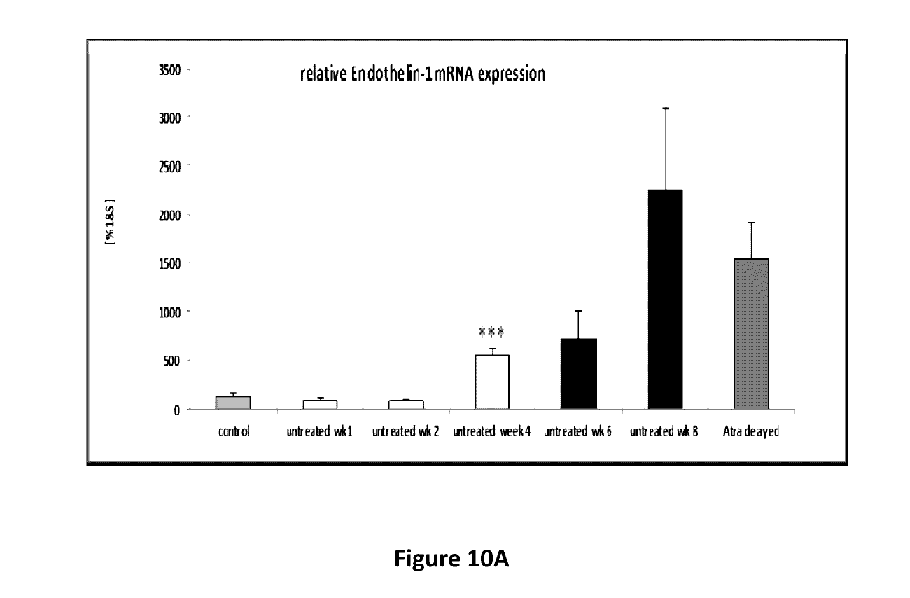

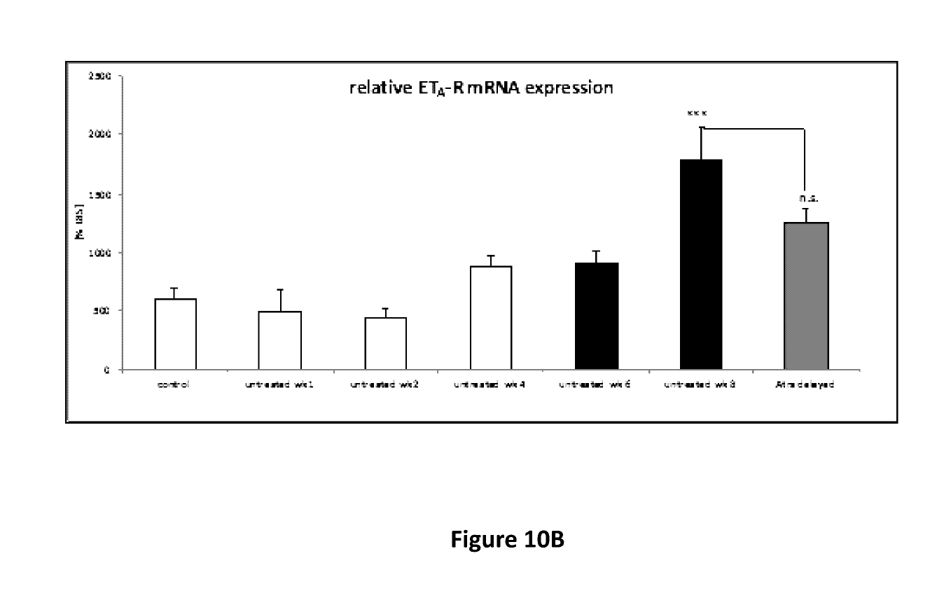

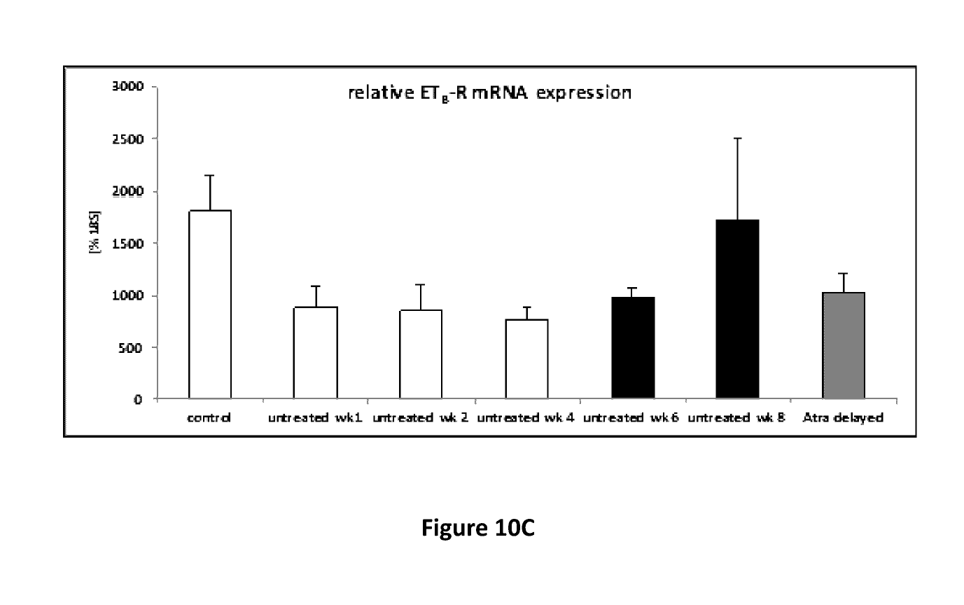

Real time investigations of the expression of Endothelin-1, ET_{A}R and ET_{B}R by mRNA expression were measured at week 1, 2, 4, 6, and 8 weeks in untreated mice (FIG. 10) revealed that there is a late upregulation of endothelin-1, ET_{A}R and ET_{B}R in the course of disease, reaching significant from week 5 (ET-1). In Atrasentan treated animals, lower expression of endothelin-1, ET_{A}R and ET_{B}R was observed, suggesting a negative cellular feedback.

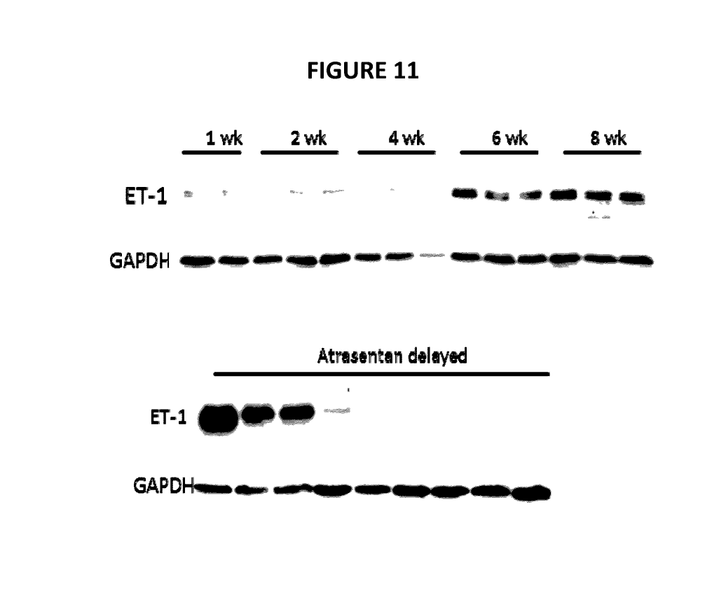

Western blot analysis was performed to confirm the observation of increased mRNA level in the late course of disease (FIG. 11). Atrasentan treated animals displayed decreased protein expression of endothelin-1. Specifically, Atrasentan was administered at the time of maximal proteinuria and strong ET-1 expression, proteinuria decreased progressively and MAP was lowered significantly (wk7: 80.4 (D) v 99.5 (U) mmHg, p=0.001). Histological evaluation (n=9) demonstrates attenuation in glomerulosclerosis (GSI: 1.69 (D) v. 2.27 (U wk 8), p=0.0008) and tubulointerstitial fibrosis (TIF & of total area 4.18 (D) v. 7.56 (U wk 8), p=0.008; podocyte numbers tend to be better preserved (podocytes per glom: 54% (D) vs. 32% (U wk 8) of healthy controls, n.s.). Of note, three of the nine animals displayed endothelin-1 expression, suggesting that there may be some individuals Atrasentan may be less effective in, and we observed that these mice had higher levels of proteinuria compared to others from this group (results not shown).

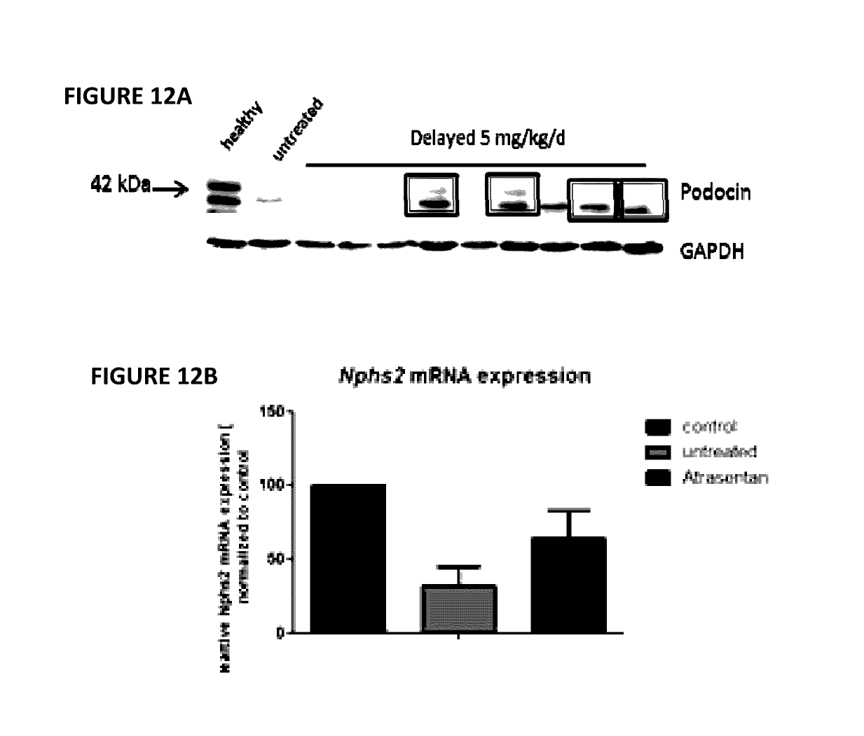

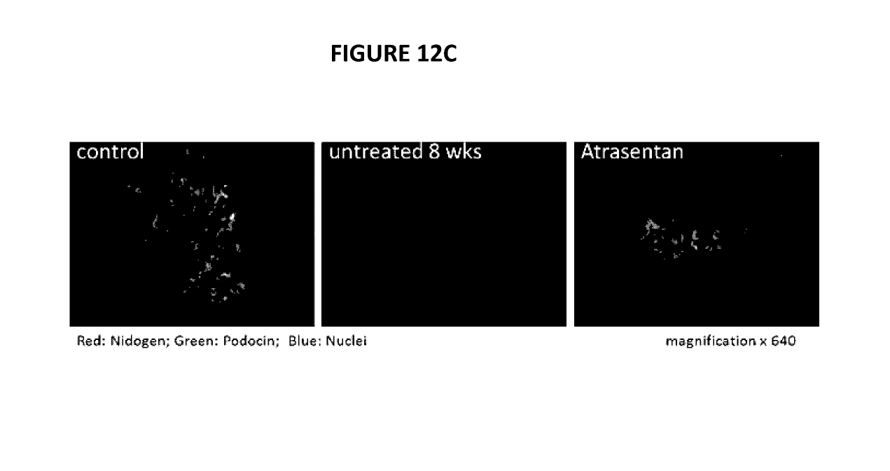

Further podocin expression analysis was carried out. Podocin protein abundance and mRNA expression was partly preserved in the treated animals. Podocin expression analysis included protein expression analysis by Western blot (FIG. 12A), mRNA expression assessed by qRT-PCR (FIG. 12B) and histological staining using immunoflurescence (FIG. 12C), demonstrating podocin expression was partly preserved in Atrasentan treated animals.

In order to investigate the course of proteinuria and the impact on survival we are filling up the group with treatment period of 8 weeks and are keeping animals for open end observation (max. 35 weeks).

Combinational Studies

In our previous studies, we found out, that RAS blockade may provide effective nephroprotection in hereditary nephropathy, delays renal failure and prolongs survival. We are performing a combined treatment with ERA and ARB (Atrasentan+Cansesartan). Not to be bound by any theory, but we believe that the combination of atrasentan and an (ACEi) or (ARBs) will provide a synergistic effect, e.g., they will provide a reduction in at least one symptom of MDS-NS greater than either treatment alone.

We claim:

1. A method of treating MDR-NS, comprising the step of administering to a pediatric subject a sufficient amount of atrasentan or a salt thereof, wherein the administration reduces, treats, improves or ameliorates one or more symptoms of MDR-NS.

2. The method of claim 1, wherein the treatment is for a period of at least 3 months.

3. The method of claim 1, wherein the treatment is for a period of at least 6 months.

4. The method of claim 1, wherein the treatment is combined with a dose of at least one of an angiotensin converting enzyme inhibitor or an angiotensin II receptor blocker.

5. The method of claim 1, wherein the pediatric subject is 6 months to onset of puberty of the subject.

6. The method of claim 5, wherein the pediatric subject is 6 months to 16 years of age.

7. The method of claim 5, wherein the pediatric subject is 6 months to 14 years of age.

8. The method of claim 5, wherein the pediatric subject is 6 months to 12 years of age.

9. The method of claim 5, wherein the pediatric subject is 6 months to 10 years of age.

10. The method of claim 5, wherein the pediatric subject is 6 months to 8 years of age.

11. The method of claim 1, wherein the pediatric subject does not respond, or responds poorly, to first-line steroid treatment or second-line drug treatments.

12. The method of claim 1, wherein the pediatric subject does not respond, or responds poorly, to first-line steroid treatment or is not considered suitable for second-line drug treatment.

13. The method of claim 1, wherein the method of administration is selected from the group consisting of oral, rectal, parenteral, intracisternal, intravaginal, intraperitoneal, topical, bucal administration and an oral or nasal spray.

14. The method of claim 1, wherein a dosage is between about 0.001 mg/kg and about 100 mg/kg daily.

15. The method of claim 14, wherein the dosage is between 0.001 mg/kg and 10 mg/kg daily.

16. The method of claim 15, wherein the dosage is between 0.01 mg/kg and 5 mg/kg daily.

17. The method of claim 1, wherein the treating of the pediatric patient in an effective amount to delay the onset or progression of end-stage renal disease (ESRD).

18. The method of claim 1, wherein reduction or delay of one or more symptoms of MSD-NS reduces glomerular hyperfiltration or reduces tubulointerstitial inflammation.

19. A method of treating MDR-NS, comprising the step of administering to a pediatric subject an effective amount of atrasentan or a salt thereof and an angiotensin-converting-enzyme inhibitors (ACEi) or angiotensin II receptor blockers (ARBs), wherein the administration reduces, treats, improves or ameliorates at least one symptom of MDR-NS.

20. The method of claim 19, wherein the ARB is candesartan, eprosartan, irbesartan, losartan, olmesartan, tasosartan, telmisartan, valsartan or combinations thereof.

21. The method of claim 19, wherein the ACEi is alacepril, benzapril, captopril, ceronapril, cilazapril, delapril, enalapril, enalaprilat, eosinopril, fosinopril, imidapril, lisinopril, moexipril, moveltipril, omapatrilat, perindopril, quinapril, ramipril, sampatrilat, spirapril, temocapril, trandolapril, or combinations thereof.

22. The method of claim 19, wherein the atrasentan or a salt thereof and the angiotensin-converting-enzyme inhibitors (ACEi) or angiotensin II receptor blockers (ARBs) have a synergistic effect in reducing, treating, improving or ameliorating at least one symptom of MDR-NS.

23. A method of reducing proteinuria in a pediatric subject suffering from MDR-NS, comprising the step of administering to the pediatric subject an effective amount of atrasentan or a salt thereof, wherein the administration reduces the level of proteinuria in the pediatric subject.

24. The method of claim 23, wherein the administration reduces the level of proteinuria in the patient by at least 10%.

25. The method of claim 24, wherein the administration reduces the level of proteinuria in the patient by at least 20%.

26. The method of claim 23, wherein the treatment reduced the level of proteinuria below 4 mg/m²/hour (or 50 mg/kg/day).

27. The method of claim 23, wherein proteinuria is measured by calculating a urine protein-to-creatinine, and wherein the urine protein-to-creatinine level is reduced at least 10%.

28. The method of claim 27, wherein the urine protein-to-creatinine ratio is less than 2.0 mg/mg.
